(19) United States
(12) Patent Application Publication
(10) Pub. No.: US 2016/0317420 A1
(43) Pub. Date: Nov. 3, 2016

(54) METHOD OF IMPROVING THE APPEARANCE OF SKIN AND COMPOSITIONS THEREFOR

(71) Applicant: The Procter & Gamble Company, Cincinnati, OH (US)

(72) Inventors: Tomohiro HAKAZAKI, Cincinnati, OH (US); Leo Timothy LAUGHLIN, II, Mason, OH (US)

(21) Appl. No.: 15/142,044

(22) Filed: Apr. 29, 2016

Related U.S. Application Data
(60) Provisional application No. 62/155,727, filed on May 1, 2015.
(60) Provisional application No. 62/162,179, filed on May 15, 2015.

(51) Int. Cl. A61K 8/60; A61K 8/06; A61Q 19/02

(52) U.S. Cl. CPC A61K 8/602; A61Q 19/02; A61K 8/064; A61K 8/062

(57) ABSTRACT

A cosmetic method for reducing glycation rate in skin by topically applying a cosmetic composition that includes an effective amount of nicotinamide riboside to a target portion of skin during a treatment period. The treatment period is sufficient for the nicotinamide riboside containing composition to improve the appearance of sallow-looking skin caused by glycation. In particular, the method enables a reduction in skin yellowness related to glycation.

### FIELD

The present disclosure is directed generally to cosmetic methods and compositions for improving the appearance of skin. More specifically, the present disclosure is directed to methods and compositions that include nicotinamide riboside for reducing the effects of advanced glycation end products on skin appearance.

### BACKGROUND

The color of normal human skin is due primarily to varying amounts and distribution of melanin, hemoglobin, and carotenoids. Of these pigments, melanin is of primary significance. But as people age, proteins in the body can become damaged, resulting sallow-looking skin. Sallow-looking skin is commonly associated with old age and sickness, and the skin often appears yellow and/or thin

One cause of sallow-looking skin is glycation. Glycation is generally recognized as a non-enzymatic process involving a monosaccharide (e.g., glucose or ribose) that reacts with an amino group of an amino acid (e.g., lysine), via the Maillard to form a Schiff base. The latter, after a molecular rearrangement sometimes referred to as the Amadori rearrangement, may lead to crosslinking of the proteins in keratinous tissue such as skin, hair and nails. This phenomenon increases regularly with age, and is characterized by the appearance of advance glycation end products (“AGEs”). Thus, there remains a need to identify cosmetic skin care agents capable of combating glycation in skin and improving the appearance of skin damaged by glycation.

Human skin is the largest organ in the human body and includes an outer epidermal layer and an underlying dermal layer. The epidermis is formed primarily from keratinocytes that undergo a constant renewal process as they migrate from the basal layer at the dermal/epidermal junction to the outmost layer of the stratum corneum. The dermis underlies and supports the epidermis, and is formed primarily of fibroblasts. Fibroblasts synthesize the extracellular matrix of the dermis, which includes collagen and elastin that provide skin with strength and elasticity.

Collagen is responsible for the strength of the dermis, and is generally resistant to breakdown, except by enzymes commonly referred to as collagenases. In the dermis, the collagen is formed as a multitude of interconnected fibrils, and it is these packed, entangled collagen fibers that provide the dermis with its structure and thickness. Collagen fibers are regularly renewed, but age and environment stressors adversely affect collagen renewal. In particular, as a person ages, the body's ability to remove AGEs decreases and eventually the rate of AGE formation outstrips the rate of collagen renewal. Additionally, some AGEs tend to have a yellowish color, and as they accumulate in dermis they can cause the skin to take on a sallow appearance, especially when the skin becomes thinner due to decreased collagen levels.

U.S. Pat. No. 6,414,038 relates to the administration of hydroxystilbenes and particularly to the topical application of 3,3′,5,5 ‘-tetrahydroxystilbene for reducing or even inhibiting the glycation of proteins, particularly proteins of the skin and/or of its related structures, thus combating skin aging. However, there is still a need to identify additional cosmetic agents for treating glycation in skin.

U.S. Pat. No. 7,666,414 relates to the topical application of compositions containing benfotiamine and pyridoxamine for the prevention and/or treatment of damage to skin, particularly skin damage resulting from reactive carbonyl species (RCS), glycation of skin proteins, formation of advanced glycation endproducts (AGEs) and formation of advanced lipoxidation endproducts (ALEs). However, there is still a need to identify additional cosmetic agents for treating glycation in skin.

PCT Pub. No. WO 2015/066382 (“Deren-Lewis”) relates to methods of using nicotinamide riboside to promote the increase of intracellular levels of nicotinamide adenine dinucleotide (NAD+) in cells and tissues for improving cell and tissue survival. Deren-Lewis discloses the use of topical nicotinamide riboside compositions for treating a variety of skin conditions by modulating the NAD+ pathway. However, Deren-Lewis does not recognize that many topical skin care compositions are aqueous and nicotinamide riboside is susceptible to hydrolysis in aqueous compositions. Thus, there is a need to ensure an effective amount of nicotinamide riboside is provided in aqueous compositions.

Accordingly, it would be desirable to provide cosmetic methods and compositions for inhibiting, reducing, or reversing the effects of glycation and advanced glycation end products on skin appearance. It would also be desirable to provide a cosmetic composition comprising an effective amount of nicotinamide riboside for inhibiting, reducing, or reversing the effects of glycation and advanced glycation end products on skin appearance.

### SUMMARY

The present disclosure relates to a cosmetic method of improving the appearance of skin exhibiting the undesirable signs of glycation. In some instances, the method comprises topically applying an effective amount of nicotinamide riboside to a target portion of skin exhibiting signs of glycation such as sallowness or yellowness during a treatment period. The treatment period is sufficient to allow the nicotinamide riboside containing composition to reduce the glycation rate in the target skin portion. In some instances, the method involves targeting a portion of skin that exhibits undesirable yellowness and applying a composition comprising an effective amount nicotinamide riboside to the target portion of skin. By reducing yellowness associated with glycation, a skin appearance benefit is provided.

### BRIEF DESCRIPTION OF THE DRAWINGS

FIG. 1 is a chart illustrating changes in glycation rates.

FIG. 2 is a chart illustrating changes in b* value.

FIG. 3 is a chart illustrating HMGB 1 released by keratinocytes.

FIG. 4 shows an image of a face with a portion of the cheek masked.

### DETAILED DESCRIPTION

It is known that niacinamide (CAS No. 98-92-0) and nicotinamide riboside, which are both NAD+ precursors, can be used to modulate the NAD+ pathway to treat cells for some of the symptoms commonly associated with aging. It is also known that niacinamide can improve the appearance of sallow skin by suppressing glycation. Prior to the present invention, it was not known whether nicotinamide riboside could provide a similar benefit, since it has been demonstrated that niacinamide and nicotinamide riboside impact skin biochemistry differently, namely biochemical pathways related to skin pigmentation, ARE modulation and glycolytic ATP depletion. It has now been discovered that nicotinamide riboside can suppress glycation in skin and reduce yellowness associated with glycation. Surprisingly, it has also been discovered that nicotinamide riboside can reduce skin yellowness in skin exposed to environmental stressors such as cigarette smoke. Without intending to be bound by theory, it is believed that nicotinamide riboside blocks an inflammatory pathway linked to the release of the High Mobility Group Box 1 (“HMGB 1”) in keratinocytes exposed to environmental stressors. HMGB 1 release is known to recruit neutrophils, which in turn are known to release factors such as reactive oxygen species, which participate in the sugar rearrangement/crosslinking reaction associated with glycation.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25 ° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

Definitions.

“Apply” or “application”, as used in reference to a composition, means to apply or spread the compositions of the present invention onto a human skin surface such as the epidermis.

“Cosmetic” means providing a desired visual effect on an area of the human body. The visual cosmetic effect may be temporary, semi-permanent, or permanent.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications. The compositions herein may optionally include one or more cosmetic agents in addition to nicotinamide riboside. In some embodiments, cosmetic agents may be incorporated in a cosmetic composition comprising a dermatologically acceptable carrier suitable for topical application to skin.

“Disposed” means an element is positioned in a particular place relative to another element.

“Effective amount” means the amount of nicotinamide riboside that is incorporated into a composition at the time the composition is made, which is sufficient to provide an anti-glycation benefit and/or yellowness reduction benefit in skin over the course of a treatment period.

“Improve the appearance of,” when used in conjunction with a skin appearance benefit, refers to reducing the sallow appearance of skin associated with glycation, which may be quantified by a decrease in b* value. An exemplary method for determining b* value is described in more detail below.

“L*a*b*” refers to the commonly recognized color space specified by the International Commission on Illumination (“CIE”). The three coordinates represent (i) the lightness of the color (i.e., L*=0 yields black and L*=100 indicates diffuse white), (ii) the position of the color between magenta and green (i.e., negative a* values indicate green while positive a* values indicate magenta) and (iii) the position of the color between yellow and blue (i.e., negative b* values indicate blue and positive b* values indicate yellow).

“Safe and effective amount” means an effective amount of nicotinamide riboside that is low enough to avoid serious side effects (within the scope of sound medical judgment).

“Skin tone agent” means a cosmetic agent intended to be applied to the skin for the purpose of effectuating a change in skin pigmentation.

“Treatment period,” as used herein means the length of time and/or frequency that a material or composition is applied to a target skin surface.

### Cosmetic Compositions

Various cosmetic compositions, and more specifically cosmetic compositions for topical application to a skin surface, are provided. These cosmetic compositions comprise a safe and effective amount of nicotinamide riboside. The cosmetic compositions herein may be provided in various product forms that include, but are not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. The cosmetic composition form may follow from the particular dermatologically acceptable carrier chosen, if present in the composition.

The cosmetic compositions herein may be made using conventional methods of making such compositions. For example, a cosmetic composition comprising an effective amount of nicotinamide riboside may be made by mixing nicotinamide riboside with a dermatologically acceptable carrier at an amount of from 0.05%, 0.5%, 1%, 2%, 3%, 4% or 5% to 20%, 15%, 10%, 8% or 6% by weight of the composition. It is known that nicotinamide riboside can undergo hydrolysis in an aqueous environment, depending on temperature and time in solution, to form niacinamide and ribose. This can be especially problematic for cosmetic compositions, which may be stored (e.g., in a warehouse, during shipping or on a store shelf) for a relatively long period of time (e.g., from 1 to 3 weeks or even from 1 to 6 months) at variety of temperatures. Typical storage temperatures for cosmetic compositions may range from 5° C. to 45° C. Thus, in order to provide an effective amount of nicotinamide riboside in an aqueous composition (i.e., a composition that includes water), it is important to base the amount of nicotinamide riboside added during manufacture on the known chemical kinetics of nicotinamide riboside and water. A discussion of the reaction kinetics of nicotinamide riboside and water can be found in a publication by Ferraz, et al., titled “Kinetic a-Deuterium Isotope Effects for Enzymatic and Nonenzymatic Hydrolysis of Nicotinamide-β-Riboside,” Archives of Biochemistry and Biophysics Vol. 191, No. 2, December, pp. 431-436, 1978.

### Nicotinamide Riboside

The methods and topical cosmetic compositions herein include a safe and effective amount of nicotinamide riboside. Nicotinamide riboside (CAS No. 1341-23-7) has the formula:

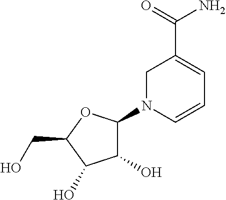 [SMILES: NC(=O)C1=CC=CN([C@@H]2O[C@H](CO)[C@@H](O)[C@H]2O)C1]

Some examples of nicotinamide riboside and heir methods of manufacture are described in U.S. Pat. No. 8,106,184. As used herein, the term “nicotinamide riboside” also includes salts of nicotinamide riboside (e.g., nicotinamide riboside hloride).

The cosmetic compositions herein include an effective amount of nicotinamide riboside. In some instances, the cosmetic compositions herein may include nicotinamide riboside at an amount of from 0.005%, 0.1%, 0.5%, 1%, 2%, 3%, 4% or 5% to :20%, 15%, 10%, 8% or 6% by weight of the cosmetic composition. It is to be appreciated that the amount of nicotinamide riboside in the present compositions may vary depending on how much of an anti-glycation benefit is desired. The foregoing amounts may be present at the time of manufacture and/or at the time of use, depending on the desired level of benefit desired. For example, the amount of nicotinamide riboside present in the composition at the time of use may be sufficient to reduce the rate of glycation, yellowness associated with glycation and/or HMGB 1 levels by at least 10%, 15%, 20%, 25%, 30%, 35%, 40%, 45%, 50%, 55%, 60%, 65%, 70%, 75%, 80%, 85%, 90%, 95% or 100%. In some instances, the safe and effective amount of nicotinamide riboside may be sufficient to reduce the level of HMGB I released in stressed keratinocytes keratinocytes exposed to a stressor such as cigarette smoke) to pre-stress levels or below, which could result in a reduction of greater than 100%. Glycation rate and changes in b* value (in vitro) can be determined according to the AGE Assay described in more detail below. HMGB1 level can be determined according to the HMGB1 Assay described below, which utilizes a conventional HMGB1 ELISA kit (e.g., the HMGB1 ELISA Kit available from IBL International as REF # ST51011) according to the manufacturer's instructions. Yellowness (i.e., b* value) may be determined in vivo according to the Imaging Method described below.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil or oil-in-water). The oil phase of the present invention may comprise silicone oils, non-silicone oils such as hydrocarbon oils, esters, ethers, and the like, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). Emulsions may also contain an emulsifier, e.g., from about 1% to about 10% or from about 2% to about 5% based on the weight of the carrier. Emulsifiers may be nonionic, anionic or cationic. Some non-limiting examples of emulsifiers are disclosed in U.S. Pat. No. 3,755,560 to Dickert et al.; U.S. Pat. No. 4,421,769 to Dixon et al.; and. McCutcheon's Detergents and Emulsifiers, North American Edition, pages 317-324 (1986).

The carrier may contain one or more dermatologically acceptable, hydrophilic diluents. As used herein, “diluent” includes materials in which the nicotinamide riboside can be dispersed, dissolved, or otherwise incorporated. Hydrophilic diluents include water, organic hydrophilic diluents such as lower monovalent alcohols (e.g., C1 -C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Optional Ingredients.

The present composition may optionally include one or more additional ingredients commonly used in cosmetic compositions (e.g., colorants, skin tone agents, skin anti-aging agents, anti-inflammatory agents, sunscreen agents, combinations of these and the like), provided that the additional ingredients do not undesirably alter the anti-glycation benefits provided by the composition. In some instances, it may be desirable to select skin tone agents that function via different biological pathways so that the actives do not interfere with one another, which could reduce the efficacy of both agents. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition. The additional ingredients, when incorporated into the composition, should be suitable for use in contact with human skin tissue without undue toxicity, incompatibility, instability, allergic response, and the like. Some nonlimiting examples of additional ingredients which may be suitable for use herein are described in U.S. Publication Nos. 2002/0022040; 2003/0049212; 2004/0175347; 2006/0275237; 2007/0196344; 2008/0181956; 2010/00092408; 2008/0206373; 2010/0239510; 2010/0189669; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533. Some further nonlimiting examples of additional ingredients, which may be particularly suitable for use in the present compositions, are provided below.

In some instances, the compositions herein can include from 0.001% to 40% (e.g., from 1% to 30%, or from 2% to 20%) of one or more particulate materials and/or cosmetic powders to provide acute look and/or feel benefits. These particulates can, for instance, be platelet shaped, spherical, elongated or needle-shaped, or irregularly shaped; surface coated or uncoated (e.g., hydrophobically coated); porous or non-porous; charged or uncharged; and can be added to the current compositions as a powder or as a pre-dispersion. For example, pigmentary-grade metal oxide particles (e.g., having an average primary particle size greater than 100 nm or from 100 nm to 500 nm) may optionally be included to provide an appearance benefit. Some nonlimiting examples of particulate materials for use herein are described in U.S. Publications Nos. 2012/0021027, 2010/0074928, 2010/0003205, 2010/0003293 and 2013/0243835.

In another example, the compositions used in accordance with the present method may include powders in the form of spherical particles, which provide an acute look and/or feel benefit. Spherical particle powders tend to improve the speed that the product appears to absorb into the skin, which helps provide increased control over product application. Spherical particle powders herein have a median particle size of from 2 μm to 40 μm, (e.g., from 3 μm to 25 μm or even from 5 μm to 15 μm). Spherical particles powders can also increase the smooth feeling of the product film on the skin. Accordingly, it may be desirable to select spherical particles that have no tackiness and a rubber hardness (as measured by Durometer A defined in JIS K 6253) in the range of 10 to 90, (e.g., 20 to 80 or even from 25 to 75). In a particularly suitable example, the composition includes 2% to 20% (e.g., 4% to 12%) spherical silicone elastomer particles or spherical starch particles. The amount of silicone elastomer powder in the composition is determined based on the particulate material being in neat form (i.e., not swollen in solvent). Some nonlimiting examples of spherical particle powders are described in co-pending U.S. Serial Nos. 14/596,360 and 14/596,374, filed by Jansen, et al., on Jan. 14, 2015.

### Mehtods of Use

The methods herein include identifying a target portion of skin (e.g., a facial skin surface such as the forehead, perioral, chin, periorbital, nose, and/or cheek) in need of treatment and/or where treatment is desired and applying a safe and effective amount of nicotinamide riboside to the target portion of skin. The nicotinamide riboside may be incorporated into a suitable cosmetic composition using conventional methods for making cosmetic compositions. Without intending to be bound by theory, it is believed that application of an effective amount of nicotinamide riboside to a target portion of skin in need of treatment can reduce glycation rate, yellowness associated with glycation, and/or HMGB 1 level over the course of a treatment period. In this way, the present compositions can improve the appearance of the target portion of skin. In some instances, the target portion of skin may not exhibit a skin health disorder associated with glycation, but a user (e.g., a relatively young user) may still wish to target such an area of skin if it is one that typically develops glycation related skin disorders later in life. In this way, the present compositions may be used as a preventative measure. In some instances, the glycation rate, b* value and/or HMGB1 level in the target area may be measured and compared to a reference amount (e.g., the corresponding value(s) in a portion of skin not typically exposed to sunlight) to determine if treatment is needed and/or desired. The composition may be applied to the target skin portion and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed.

The treatment period is ideally of sufficient time for the nicotinamide riboside containing composition to improve the appearance of the target portion of skin, which may correspond to a reduction in glycation rate, yellowness associated with glycation, and/or HMGB1 level. The treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a sallow portion of skin) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Test Methods

### Imaging Method

This method provides a means for capturing a reproducible and analyzable image for determining L*a*b* values, and b* value in particular. It is to be appreciated that any suitable image capture device along with imaging software and other associated ancillary equipment (e.g., computer and lights) which are equivalent to those described in this method may be used. The imaging system in this method incorporates a FUJI-52 Pro brand CCD SLR digital camera which delivers a 6 megapixel uncompressed image (BMP) and a raw image file (RAF). Prior to taking a photograph, the test subject is illuminated with a JTL 1000W flash through two linear polarizers in crossed axis orientation. A chart containing Munsell Color Standard Neutral N2-N9.5 is captured in every image for standardization and color correction purposes.

In preparation for image capture, test subjects are required to wash their faces and wait for at least 15 minutes to let their face dry. The hair of the subject is covered with a hairnet and the head and shoulders of the subject are covered with a black cloth. All jewelry that can be seen in an image area of interest is removed. The subjects are equilibrated in a control room at 20 -25 ° C. and 40 -60% relative humidity for 30 minutes. Next, each subject is suitably positioned, in front of the camera and one or more images of each side of the face are captured. The captured image(s) are then processed by converting the raw image to a .jpg file format.

Next, the .jpg format image is analyzed by a computer with suitable image analysis software. In some instances, it may be desirable to analyze only a portion of the image (i.e., a region of interest (“ROI”)). The ROI may be “masked,” for example, as shown in FIG. 4, using image editing software such as Photoshop® or Image J® brand software. The masked region (e.g., cheek 100 in FIG. 4) can then be isolated and analyzed as a separate image. It is to be appreciated that the image need not necessarily be masked for suitable analysis, and in some instances the entire image may be analyzed. In some instances, it may be desirable to reduce the size of the image, mask and/or region of interest by several pixels (e.g., between 5 and 15 pixels) around the outer edge of the image where some shadowing may occur.

Since color may be perceived as being relative, depending on, for example, which instruments and/or imaging system is used, it can be important to color correct the image or region of interest for each subject using a suitable color correction technique (e.g., according to International Color Consortium standards and practices), which helps make the color determination by the system less instrument specific. The RGB values in the captured images, which are device dependent, are converted to L*a*b* values. The L*a*b* values can be calculated using a suitable RGB conversion tool (e.g., software installed on the computer or a suitable conversion tool found online) The conversion from RGB values to L*a*b* values can be performed on the entire image, a ROI or on one or more individual pixels. The resulting L*a*b* values may be averaged to provide average values for the image or a region of interest.

### EXAMPLES

### Example 1

Table 1 provides examples of cosmetic composition formulas suitable for use with the methods herein. The cosmetic compositions are made by blending the A phase components with a suitable mixer (e.g., Tekmar RW2ODZM) and heating to a temperature of 70-80 ° C. and maintaining the temperature while stirring. Separately, blend the B phase components with a suitable mixer and heat to 70 -75 ° C., maintaining temperature while mixing. Phase B is added to Phase A while mixing well to emulsify. The emulsion is then milled using a suitable mill (e.g., Tekmar T-25) for 5 minutes. When the emulsion is at 60 ° C., phase C is added while continuing to mix. At 40 ° C., the ingredients of phase D and E are added to the emulsion. The emulsion is then milled using a suitable mill (Tekmar T-25) for 5 minutes resulting in a uniform product.

TABLE 1

| | 1 | 2 | 3 | 4 | 5 | 6 |
|---|---|---|---|---|---|---|
| Component | % | | | | | |
| Phase A | | | | | | |
| water | qs | qs | qs | qs | qs | qs |
| glycerol | 5.00 | 7.00 | 3.00 | 10.00 | 5.00 | 15.00 |
| disodium EDTA | 0.10 | 0.05 | 0.10 | 0.10 | 0.05 | 0.10 |
| Phase B | | | | | | |
| Isopropyl Isostearate | 5.00 | 2.50 | 1.33 | 2.50 | 5.00 | 2.50 |
| Isohexadecane | 1.00 | 1.50 | 3.00 | 1.00 | 3.00 | 5.00 |
| Distearyldimonium Chloride | 0.00 | 0.50 | 1.00 | 1.50 | 0.00 | 1.50 |
| Steareth-2 | 0.50 | 2.00 | 1.00 | 1.00 | 1.50 | 3.00 |
| cetyl alcohol | 0.25 | 0.50 | 0.32 | 0.50 | 1.00 | 0.40 |
| tocopherol acetate | 0.00 | 0.50 | 0.50 | 0.50 | 0.25 | 1.00 |
| Steareth-21 | 0.50 | 1.00 | 0.40 | 0.80 | 1.25 | 2.00 |
| stearyl alcohol | 0.70 | 1.50 | 2.00 | 2.25 | 3.00 | 4.50 |
| behenyl alcohol | 0.80 | 1.00 | 0.40 | 0.60 | 1.50 | 0.60 |
| ethyl paraben | 0.20 | 0.20 | 0.20 | 0.20 | 0.20 | 0.20 |
| propyl paraben | 0.10 | 0.10 | 0.10 | 0.10 | 0.10 | 0.10 |
| polymethylsilsesquioxane | 1.25 | 2.50 | 2.00 | 0.50 | 0.25 | 1.50 |
| Phase C | | | | | | |
| Polyethylene | 1.50 | 1.00 | 1.50 | 2.00 | 1.25 | 1.00 |
| Phase D | | | | | | |
| Water | 5.00 | 10.00 | 10.00 | 5.00 | 10.00 | 15.00 |
| Nicotinamide Riboside | 2.00 | 5.00 | 5.00 | 2.50 | 4.00 | 7.00 |
| Chloride¹ | | | | | | |
| dexpanthenol | 0.25 | 0.50 | 0.50 | 2.00 | 1.00 | 2.00 |
| Phase E | | | | | | |
| benzyl alcohol | 0.25 | 0.25 | 0.25 | 0.25 | 0.25 | 0.25 |
| dimethicone/dimethiconol | 0.5 | 1.00 | 2.00 | 0.25 | 2.00 | 2.00 |

¹available from Chromadex, Irvine CA

### Example 2

### In Vitro Suppression of AGE Formation and Yellowness Reduction—AGE Assay.

This example utilizes an AGE Assay to demonstrate the ability of nicotinamide riboside (“NR”) to suppress AGE formation as compared to a positive control, a negative control and niacinamide, which is a benchmark skin tone agent known to suppress glycation.

AGE Assay

The anti-glycation ability of NR was determined as follows. Samples were prepared in a 96-well plate (3 replicates each) at a total volume of 250 μl/well. The test samples contained 112.5 μl of 2% w/v gelatin solution (commercially available from Sigma as Catalog # G1393-100 ML), 10 μl of 1 M glyceraldehyde (commercially available from Sigma as Catalog # G5001-1 G), 102.5 p1 Dulbecco's phosphate buffered saline (“DPBS”) (commercially available from Gibco's), and varying doses (0.1%, 0.5%, 1% and 5% w/v) of nicotinamide riboside chloride (“NR”) (available from Chromadex, Irvine, CA). The positive control contained 112.5 μl gelatin, 10 μl of 1 M glyceraldehyde and 127.5 μl DPBS. The negative control contained 112.5 μl gelatin and 137.5 μl DPBS. A 5% w/v niacinamide (“N”) composition was used for a benchmark comparison. The niacinamide sample contained the same amounts of gelatin, glyceraldehyde and DPBS as the 5% NR sample. The plate containing the test samples was placed in a CO₂ incubator (e.g., THERMO SCIENTIFIC FORMA brand CO₂ incubator available from Fisher Scientific, Waltham, Mass. or equivalent) and incubated for 24 hours at 37° C.

After 24 hours, the plate containing the test samples was removed from the incubator and placed in a SPECTRAMAX Plus brand spectrophotometer (available from Molecular Devices, Sunnyvale, Calif.). The spectrophotometer was set to detect fluorescence intensity (“FLR”) at 400/465 nm (ex/em). Glycation rate was determined by measuring FLR, at 0-hour (i.e., at the start of the test), 2-hour, 5-hour and 24-hour time points. The change in fluorescence intensity (“ΔFLR”) was determined by comparing the FLR of a well at a sampling time point to the 0-hour time point. To determine yellowness (i.e., b* value), a SPECTRAMAX Plus brand spectrophotometer was used to collect wavelength/absorbance pairs from 350 nm to 750 nm in 10 nm increments following the same time course as the FLR measurements (i.e., 0-hour, 2-hour, 5-hour and 24-hour). The wavelength/absorbance pairs were then converted to L*a*b* values by a computer using conversion software. The change in yellowness (“Ab*”) at a particular time point was determined by subtracting the b* value at the 0-hour time point from the b* value measured at the time point of interest. To correct for yellowness that is not due to the glycation reaction between the gelatin and glyceraldehyde, a blank well was set up for each treatment leg. The blank well contains every ingredient in the corresponding treatment leg except for the gelatin. The b* value of the blank well is subtracted from the b* value measured for each well before calculating the final Ab* value.

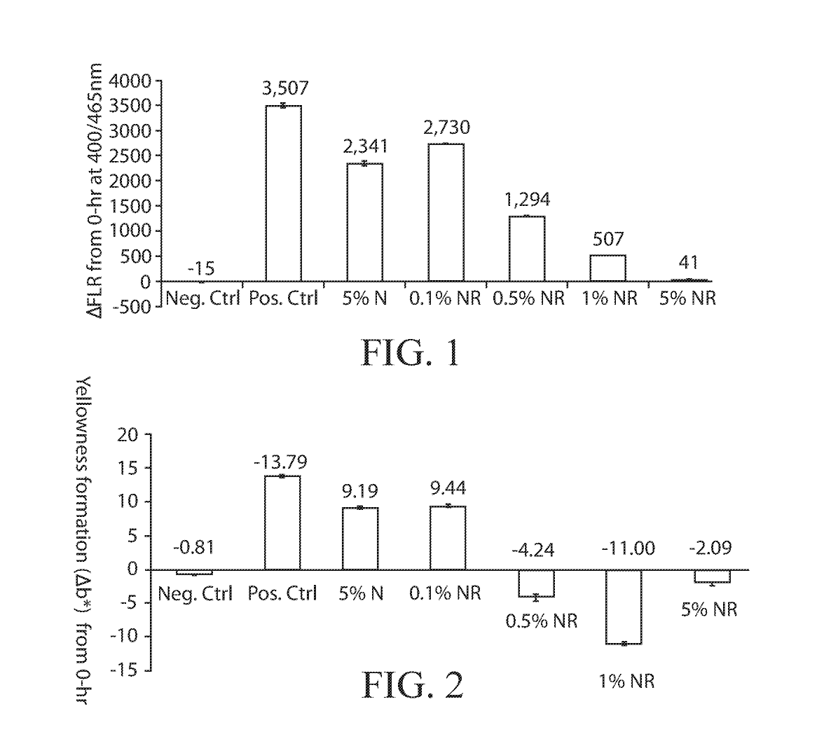

The ΔFLR at the 24-hour time point is illustrated in Table 2 and FIG. 1. As shown in Table 2 and FIG. 1, the nicotinamide riboside was able to reduce glycation rate relative to the positive control, and at concentrations of 0.5% and greater performed better than the niacinamide benchmark.

TABLE 2

| | ΔFLR at | | % reduction in | | |
|---|---|---|---|---|---|
| | 24-hr vs. | Standard | glycation rate vs. | p-value vs. | p-value |
| Treatment Leg | 0-hr | Deviation | pos. ctrl. | pos. ctrl. | vs. N |
| Negative Control | −15 | 4.416 | — | <0.05 | <0.05 |
| Positive Control | 3507 | 40.859 | — | — | <0.05 |
| 5% N | 2341 | 33.101 | 33.2 | <0.05 | — |
| 0.1% NR | 2729 | 12.51 | 22.2 | <0.05 | <0.05 |
| 0.5% NR | 1294 | 11.978 | 63 | <0.05 | <0.05 |
| 1% NR | 507 | 3.250 | 85.5 | <0.05 | <0.05 |
| 5% NR | 41 | 1.919 | 98.8 | <0.05 | <0.05 |

The Δb* at the 24-hour time point is shown in Table 3 and FIG. 2. As shown in Table 3 and FIG. 2, the nicotinamide riboside was able to reduce yellowness relative to the positive control, and at concentrations of 0.5% and greater even performed better than the niacinamide benchmark. At concentrations of 0.5% and higher, the nicotinamide riboside was able to reduce b* values below the starting values (i.e., b* value measured at 0-hour), as indicated by the negative change in b* value.

TABLE 3

| | Δb* at | | | |
|---|---|---|---|---|
| | 24-hr vs. | Standard | p-value vs. | p-value |
| Treatment Leg | 0-hour | Deviation | pos. ctrl. | vs. N |
| Negative Control | −0.81 | 0.054 | <0.05 | <0.05 |
| Positive Control | 13.79 | 0.179 | — | <0.05 |
| 5% N | 9.19 | 0.109 | <0.05 | — |
| 0.1% NR | 9.44 | 0.349 | <0.05 | 0.303 |
| 0.5% NR | −4.24 | 0.409 | <0.05 | <0.05 |
| 1% NR | −11.00 | 0.438 | <0.05 | <0.05 |
| 5% NR | −2.09 | 0.190 | <0.05 | <0.05 |

### Example 3

### In Vitro Stress Test—HMGB1 Assay

This example demonstrates the ability of nicotinamide riboside to reduce the amount of HMGB 1 released from keratinocytes subjected to stress from cigarette smoke extract (“CSE”).

HMGB1 Assay

Human neonatal keratinocytes (available from Thermo) were placed in each well of four 12-well plates. Each well also contained 2 ml of EPILIFE brand keratinocyte medium. The plates were incubated at 37 ° C. in a CO₂ incubator until cell confluency reached 70%. Once the cell confluency reached 70%, the keratinocyte medium in each well was replaced with the appropriate medium for the test (i.e., control medium, CSE test medium, niacinamide (“N”)+CSE test medium or nicotinamide riboside chloride (“NR”)+CSE test medium) to produce test samples. The resulting test samples were incubated for 24 hours at 37 ° C. in a CO₂ incubator, after which the medium in each cell was removed and the HMGB 1 levels were determined using an ELISA kit from IBL according to the manufacturer's instructions.

The N and NR media were made by adding either niacinamide or nicotinamide riboside chloride (available from Chromadex, Irvine, Calif.) to EPILIFE brand keratinocyte medium to produce a 0.01% w/v solution. The cigarette smoke extract was made by suctioning a lit cigarette through a vacuum flask containing 60 mL of phosphate buffered saline (“PBS”). 4 CAMEL brand cigarettes with the filters cut off were used to create a batch of CSE. The CSE was diluted 1:20 using keratinocyte medium to create the CSE test medium. The N+CSE medium and NR+CSE medium were prepared by adding CSE test medium to final dilution of 1:6 (CSE:medium) and either N or NR to final dilution of 0.01% w/v. Unmodified keratinocyte medium was used as the control.

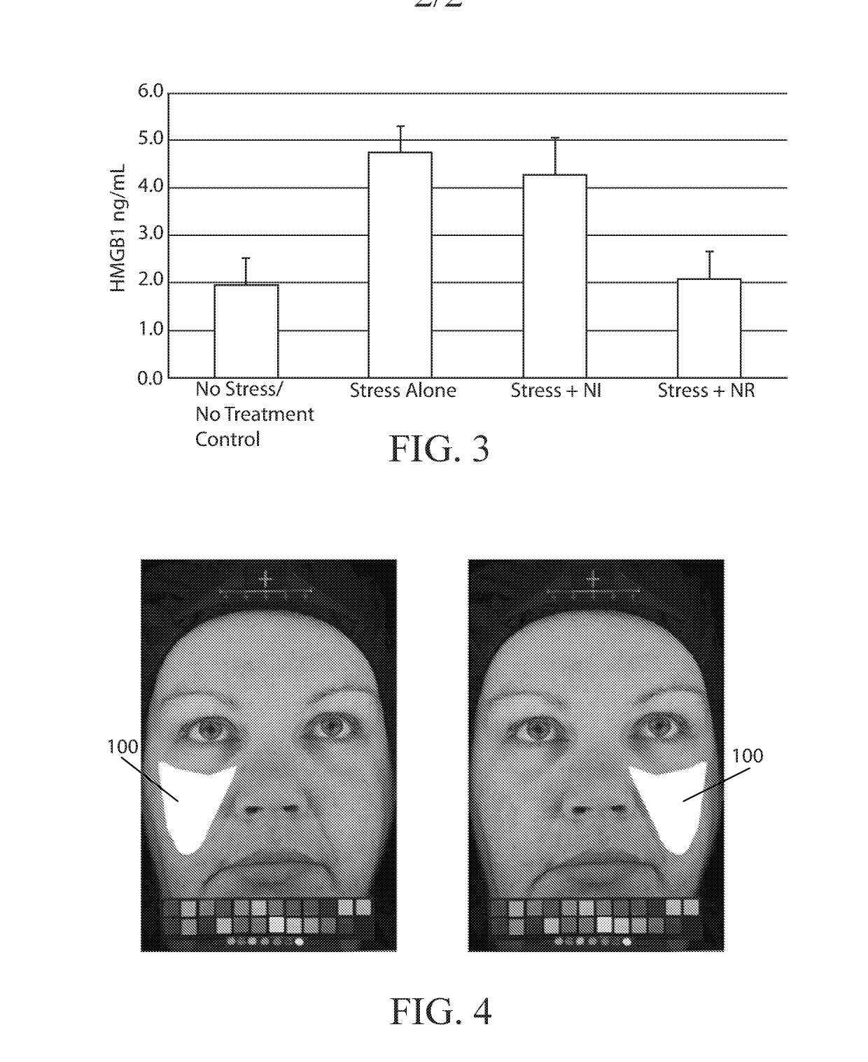

Table 4 and FIG. 3 illustrate the results of the test. P-values of less than 0.1 are considered significant. As shown in Table 4 and FIG. 3, the CSE medium caused an increase in the amount of HMGB 1 released compared to the control, which was expected. The addition of niacinamide appears to have no significant effect on the amount of HMGB 1 released by keratinocytes exposed to CSE, relative to the CSE test medium without niacinamide. Surprisingly, NR appears to significantly reduce HMGB1 released by keratinocytes exposed to CSE, and even returns HMGB 1 level to almost the same level as the control. This test demonstrates that nicotinamide riboside can successfully inhibit HMGB1 release from keratinocytes exposed to a stressor such as cigarette smoke, and thus may reduce glycation in skin associated with such stressors.

TABLE 4

| | No Stress | | | |
|---|---|---|---|---|
| Treatment | (control) | CSE | CSE + N | CSE + NR |
| AVG HMGB1 | 1.95 | 4.7319 | 4.2790 | 2.0741 |
| released (ng/ml) | | | | |
| p-value vs. control | 1 | 0.000179 | 0.0016 | 0.7536 |
| p-value vs. CSE | 0.000179 | — | 0.3198 | 8.0598E−05 |
| Std Dev | 0.29 | 0.25 | 0.34 | 0.26 |

### Example 4

### Clinical Study

This example demonstrates the ability of a cosmetic composition comprising nicotinamide riboside to reduce yellowness in skin. Composition #3 from Table 1 was used in this study. Approximately four months passed from the time the composition was made to the time it was tested. During this time, the composition was stored at about room temperature (i.e., ˜25 C).

The clinical study in this example is a 9-week, randomized, double-blinded, split-face, round robin study, which includes a 1 week normalization period and an 8 week test product usage period. The cosmetic compositions tested in the clinical study included a test composition comprising nicotinamide riboside (i.e., Composition #3 from Table 1) and the control composition set forth in Table 5. The control composition was made using conventional methods known in the art for making such compositions and was made using conventional methods known in the art for making such compositions.

TABLE 5
Control Composition

| Component | % |
|---|---|
| Phase A | |
| water | qs |
| glycerol | 3.00 |
| disodium EDTA | 0.10 |
| Phase B | |
| Isopropyl Isostearate | 1.33 |
| Isohexadecane | 3.00 |
| cetearyl glucoside | 0.20 |
| cetyl alcohol | 0.32 |
| tocopherol acetate | 0.50 |
| PEG-l00 stearate | 0.10 |
| stearyl alcohol | 0.48 |
| behenyl alcohol | 0.40 |
| ethyl paraben | 0.20 |
| propyl paraben | 0.10 |
| polymethylsilsesquioxane | 0.25 |
| Phase C | |
| polyacrylamide/C13-14 | 2.00 |
| isoparaffin/laureth-7 | |
| Phase D | |
| benzyl alcohol | 0.25 |
| dimethicone/dimethiconol | 2.00 |

Asian females aged 25 to 55 years old and having relative dark skin tone (L*<60, by Chromameter CR400) were selected to participate in the study. Prior to application of a test or control composition, the test subjects washed their face with OLAY DEEP PURIFY CLEANSER brand facial cleanser. After washing, the test product was applied to one side of the test subject's face, and the vehicle control was applied to the other side of the subject's face. This was done twice per day (morning/evening) during the test period. Dosage was 0.5 g per split face (forehead to jawline 4mg/cm²). Measurements were taken at the start of the test period (baseline) and after 2, 4 and 8 weeks of treatment. Digital images were captured and analyzed for changes in b* value according the Imaging Method described above. The data were statistically analyzed with a known Mixed Model (e.g., available from SAS Institute, Cary, NC, U.S.A.) for repeated measures with the subject effect fitted as random, and the other effects (treatment, side (left and right), week, treatment-by-week interaction, age, baseline) fitted as fixed. Values are considered statistically significant if the p-value is less than or equal to 0.05.

The results of the clinical study are illustrated in Table 6. Table 6 shows the change in yellowness values (Ab*) for the test composition relative to the control and baseline values at weeks 2, 4, and 8 for each composition. Baseline values for all test subjects were measured on Day 0 and averaged to provide a common baseline for use in the test. As shown in Table 6, treatment with the test composition reduced yellowness (i.e., provided negative Ab* value) relative to the baseline value at weeks 4 and 8, and reduced yellowness more than the control at weeks 2, 4 and 8.

TABLE 6

| | | | | Δb* from | | | |
|---|---|---|---|---|---|---|---|
| | N | Composition | b* Value | Baseline | p-value | Δb* from Control | p-value |
| Baseline | 41 | — | 21.454 | — | — | — | — |
| Week 2 | 41 | Vehicle | 21.645 | 0.191 | 0.0012 | — | — |
| Week 4 | 41 | Control | 21.283 | −0.171 | 0.0214 | — | — |
| Week 8 | 41 | | 21.537 | 0.083 | 0.2703 | — | — |
| Week 2 | 41 | Test | 21.447 | −0.006 | 0.9303 | −0.198 | 0.0120 |
| Week 4 | 41 | Composition | 21.028 | −0.426 | <0.0001 | −0.255 | 0.0048 |
| Week 8 | 41 | (5% NR) | 21.166 | −0.288 | <0.0001 | −0.370 | <0.0001 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic method of reducing glycation rate in skin, comprising:
a. identifying a target portion of skin where a reduction in glycation rate is desired; and
b. topically applying a cosmetic composition to the target portion of skin during a treatment period, wherein the cosmetic composition comprises an effective amount of nicotinamide riboside, and the treatment period is sufficient to reduce glycation rate.

2. The method of claim 1, wherein the glycation rate is reduced by at least 10%.

3. The method of claim 1, wherein, prior to the treatment period, the target portion of skin has a glycation rate that is greater than a glycation rate of a portion of skin not typically exposed to sunlight.

4. The method of claim 1, wherein the target portion of skin comprises a sallow appearance.

5. The method of claim 1, wherein the treatment period is for at least 2 weeks.

6. The method of claim 1, wherein the nicotinamide riboside is present at an amount of from about 0.1% to about 10%.

7. The method of claim 1, wherein the cosmetic composition comprises a dermatologically acceptable carrier that includes one or more hydrophilic diluents.

8. The method of claim 1, wherein cosmetic composition comprises a dermatologically acceptable carrier in the form of a water-in-oil emulsion.

9. The method of claim 1, wherein the effective amount of nicotinamide riboside reduces the level of HMGB1 released from keratinocytes exposed to a stressor by at least 10%.

10. The method of claim 9, wherein the stressor is cigarette smoke extract or ultraviolet radiation.

11. A method of reducing yellowness in skin related to glycation, comprising:
a. identifying a target portion of skin where reduced yellowness is desired; and
b. topically applying a cosmetic composition to the target portion of skin during a treatment period, wherein the cosmetic composition is formulated with about 0.005% to about 20% nicotinamide riboside and the treatment period is sufficient to reduce yellowness in the target portion of skin.

12. The method of claim 11, wherein the yellowness reduction corresponds to a reduction in b* value.

13. The method of claim 12, wherein the effective amount of nicotinamide riboside reduces b* value by at least 1 according to the AGE Assay.

14. The method of claim 11, wherein the cosmetic composition comprises a dermatologically acceptable carrier in the form of an oil-in-water emulsion.

15. The method of claim 11, wherein the cosmetic composition comprises at least one additional cosmetic agent.

16. A method of making an aqueous cosmetic composition for improving the appearance of skin, comprising:
a. mixing an effective amount of nicotinamide riboside with a dermatologically acceptable carrier, wherein the dermatologically acceptable carrier includes water and the effective amount of nicotinamide riboside is determined from the chemical kinetics of the nicotinamide riboside and the water at a temperature of between 5° C. and 45° C.; and
b. storing the cosmetic composition for at least 1 week at a temperature of between 5 ° C. and 45 ° C. prior to use by a user.

17. The method of claim 16, wherein the effective amount of nicotinamide riboside provides at least one of a reduction in b* value and a reduction glycation rate.
